(19) United States
(12) Patent Application Publication
(10) Pub. No.: US 2022/0402858 A1
(43) Pub. Date: Dec. 22, 2022

(54) METHOD FOR PREPARING DIAMINOBIPHENYL COMPOUND

(71) Applicant: SEIKA CORPORATION, Wakayama (JP)

(72) Inventors: Takahiko Tatsumi, Wakayama (JP); Motonori Takeda, Wakayama (JP); Kazuhide Nishiyama, Wakayama (JP); Shinichi Kitahara, Wakayama (JP)

(21) Appl. No.: 17/783,848

(22) PCT Filed: Dec. 2, 2020
(86) PCT No.: PCT/JP2020/044817

§ 371 Date: Jun. 9, 2022

(30) Foreign Application Priority Data
Dec. 26, 2019 (JP) 2019-237065
Nov. 5, 2020 (JP) 2020-184837

(51) Int. Cl. C07C 209/54; B01J 27/02

(52) U.S. Cl. CPC C07C 209/54; B01J 27/02

(57) ABSTRACT

The object of the present invention is to provide a method which provides a diaminobiphenyl compound in a high yield and in a short period of time by a benzidine rearrangement reaction of a hydrazobenzene having a bulky substituent group at the meta position. Specifically, the present invention provides a method for preparing a diaminobiphenyl compound represented by the following formula (1):

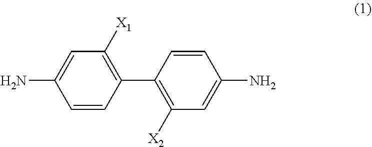

wherein, X₁ and X₂ are, independently of each other, a group selected from the group consisting of a trifluoromethyl group and, optionally fluorinated, isopropyl, isobutyl, sec-butyl, tert-butyl and neopentyl groups, comprising a step of subjecting a diphenylhydrazine compound represented by the following formula (2)

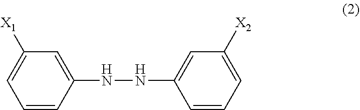

wherein X₁ and X₂ are as defined above,
to a benzidine rearrangement reaction in the presence of an organic solvent and an inorganic acid at a temperature of from −70° C. to −11° C.
to obtain the diaminobiphenyl compound represented by the formula (1).

### FIELD OF THE INVENTION

The present invention relates to a method for preparing a diaminobiphenyl compound efficiently and at a high yield by a benzidine rearrangement reaction of a hydrazobenzene having a bulky substituent group at the meta position. A particularly preferred embodiment relates to a method for subjecting 3,3′-bis(trifluoromethyl)hydrazobenzene having a trifluoromethyl group at the meta position to a benzidine rearrangement reaction to produce 2,2′-bis(trifluoromethyl)-4,4′-diaminobiphenyl efficiently and at a high yield.

### BACKGROUND OF THE INVENTION

It is known that 2,2′-bis(trifluoromethyl)-4,4′-diaminobiphenyl (hereinafter, referred to as TFMB) represented by the following formula (d) is produced by subjecting hydrazobenzene to a benzidine rearrangement reaction. For example, m-nitrobenzotrifluoride represented by the following formula (a) as a raw material is reduced to produce 3,3′-bis(trifluoromethyl) azobenzene (hereinafter, referred to as the azo compound) represented by the following formula (b), which is reduced to produce 3,3′-bis(trifluoromethyl) hydrazobenzene (hereinafter, referred to as the hydrazo compound) represented by the following formula (c) which is then subjected to a benzidine rearrangement reaction to produce TFMB represented by the following formula (d) (Non-Patent Literatures 1 and 2).

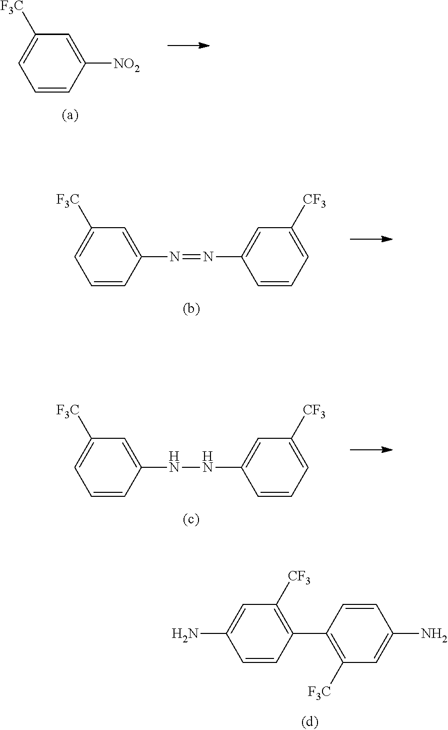 [SMILES: O=[N+]([O-])c1cccc(C(F)(F)F)c1 ; FC(F)(F)c1cccc(N=Nc2cccc(C(F)(F)F)c2)c1 ; FC(F)(F)c1cccc(NNc2cccc(C(F)(F)F)c2)c1 ; Nc1ccc(-c2ccc(N)cc2C(F)(F)F)c(C(F)(F)F)c1]

In the method described in Non-Patent Literature 1, a benzidine rearrangement reaction is caused by dissolving the hydrazo compound in ethanol, to which an ethanol solution in concentrated hydrochloric acid is dropped at 0° C. to produce TFMB. However, the yield of TFMB obtained by the method is 17%, based on the hydrazo compound. In the method described in Non-Patent Literature 2 a benzidine rearrangement reaction is caused by dissolving the hydrazo compound in alcohol and dropped into an aqueous sulfuric acid to produce TFMB. However, the yield of TFMB obtained by the method is 10%, based on the hydrazo compound.

Patent Literature 1 describes a method for preparing 2,2′-bis(trifluoromethyl)-4,4′-diaminobiphenyl (TFMB), in which the rearrangement is carried out by dropping a solution of 3,3′-bis(trifluoromethyl) hydrazobenzene in a water-immiscible organic solvent into an inorganic acid. The reaction temperature during the preparation method is 0 to 80° C. and the reaction time is 2 to 10 hours. The yield of TFMB is 25.4 to 31.8%, based on m-nitrobenzotrifluoride.

Patent Literature 2 describes a method wherein m-nitrobenzotrifluoride is reduced using 3 to 8 mols of zinc, in a nitrogen atmosphere, in the presence of an organic solvent selected from a water-immiscible organic solvent, an alcohol or a mixture of a water-immiscible organic solvent and an alcohol and an alkaline aqueous solution to directly produce 3,3′-bis(trifluoromethyl) hydrazobenzene which is dropped to an organic solvent in the presence of an inorganic acid to cause a rearrangement reaction to produce 2,2′-bis(trifluoromethyl)-4,4′-diaminobiphenyl (TFMB). The reaction temperature of the benzidine rearrangement reaction in the method described in Patent Literature 2 is −10 to +80° C. and the reaction time is 2 to 10 hours. The yield of TFMB is 24.6 to 32.1%, based on m-nitrobenzotrifluoride.

### PRIOR LITERATURES

### Patent Literatures

[Patent Literature 1] Japanese Patent No. 4738345
[Patent Literature 2] Japanese Patent No. 4901174

### Non-Patent Literatures

[Non-Patent Literature 1] Journal of Polymer Science: Part A: Polymer Chemistry, Vol. 37, pages 937 to 957 (1999)
[Non-Patent Literature 2] Journal of Chemical Society, pages 1994 to 1998 (1953)

### SUMMARY OF THE INVENTION

### Problems to be Solved by the Invention

The yield of TFMB in the methods described in Patent Literature 1 and 2 is in the range of 24 to 32% based on the m-nitrobenzotrifluoride, and these yields are not satisfactory. If there is a bulky substituent group such as a trifluoromethyl group at the meta position of the benzene in the benzidine rearrangement reaction, the selectivity of the rearrangement reaction decreases, and therefore, there is difficulty in obtaining TFMB in a high yield by the conventional preparation methods. The benzidine rearrangement reaction generally has a fast reaction rate and a high generation of heat during the reaction. In order to increase the selectivity, the reaction is carried out over a long time by dropping the reactant while removing heat as described in Patent Literatures 1 and 2. Thus, it requires a long period of time such as 2 to 10 hours.

Therefore, the object of the present invention is to provide a method which provides a diaminobiphenyl compound in a high yield and in a short period of time by a benzidine rearrangement reaction of a hydrazobenzene having a bulky substituent group at the meta position. In particular, the object of the present invention is to provide a method for preparing 2,2′-bis(trifluoromethyl)-4,4′-diaminobiphenyl efficiently and at a high yield by subjecting a 3,3′-bis(trifluoromethyl) hydrazobenzene having a trifluoromethyl group at the meta position to a benzidine rearrangement reaction.

### Means to Solve the Problems

That is, the present invention provides a method for preparing a diaminobiphenyl compound represented by the following formula (1):

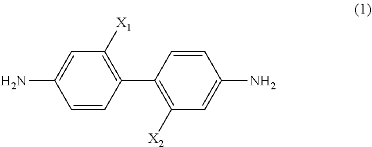

wherein X₁ and X₂ are, independently of each other, a group selected from the group consisting of a trifluoromethyl group and, optionally fluorinated, isopropyl, isobutyl, sec-butyl, tert-butyl and neopentyl groups,

comprising a step of subjecting a diphenylhydrazine compound represented by the following formula (2):

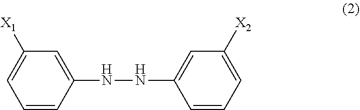

wherein X₁ and X₂ are as defined above,
to a benzidine rearrangement reaction in the presence of an organic solvent and an inorganic acid at a temperature of from −70° C. to −11° C.
to obtain the diaminobiphenyl compound represented by the formula (1).
Preferably, the present invention provides the preparation method wherein the benzidine rearrangement reaction is carried out at a temperature from −60° C. to −20° C.

A preferred embodiment of the present invention provides the preparation method in which the benzidine rearrangement reaction is carried out in the presence of an additive which prevents solidification of a reaction mixture and/or improves the flowability of the reaction mixture. A more preferred embodiment provides the preparation method in which the benzidine rearrangement reaction is carried out at a temperature of from −70° C. to −45° C., more preferably at a temperature of from −55° C. to −50° C. in the presence of the additive.

### Effects of the Invention

According to the preparation method of the present invention, a diaminobiphenyl compound is obtained in a high yield by a benzidine rearrangement reaction of a hydrazobenzene having a bulky substituent group at the meta position. In particular, by carrying out the benzidine rearrangement reaction in the presence of an additive which will be described later, a diaminobiphenyl compound having a bulky substituent group at the meta position, in particular, a 2,2′-bis(trifluoromethyl)-4,4′-diaminobiphenyl, is produced in an extremely short reaction time of from 1 minute to 1 hour and in a high yield. This is preferable because the productivity is further improved.

### DETAILED DESCRIPTION OF THE INVENTION

The preparation method of the present invention will be described below in detail.

The present invention is a method for preparing a diaminobiphenyl represented by the following formula (1).

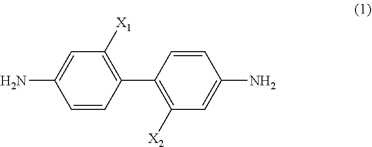

In the preparation method of the present invention, the benzidine rearrangement reaction is carries out by subjecting a diphenylhydrazine compound represented by the following formula (2) to the benzidine rearrangement reaction at a temperature from −70° C. to −11° C. to thereby produce the diaminobiphenyl represented by the formula (1).

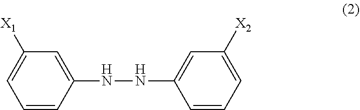

In formulas (1) and (2), X₁ and X₂ are, independently of each other, a group selected from the group consisting of a trifluoromethyl group and, optionally fluorinated, isopropyl, isobutyl, sec-butyl, tert-butyl and neopentyl groups. The diphenylhydrazine compound having these groups are reacted at a high conversion rate by the preparation method of the present invention, and a diaminobiphenyl compound is obtained in a high yield. Preferably, X₁ and X₂ are a trifluoromethyl group. The compound in which X₁ and X₂ are a trifluoromethyl group is the 2,2′-bis(trifluoromethyl)-4,4′-diaminobiphenyl represented by the following formula.

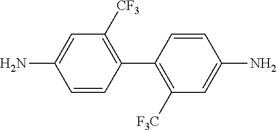 [SMILES: Nc1ccc(-c2ccc(N)cc2C(F)(F)F)c(C(F)(F)F)c1]

The preparation method of the present invention carries out the rearrangement reaction at a temperature from −70° C. to −11° C. The upper limit is preferably −18° C. lower, more preferably −20° C. lower, even more preferably −30° C. lower, even more preferably −40° C. lower, even more preferably −45° C. lower, and most preferably −50° C. lower. The lower limit of the reaction temperature is −70° C. higher, preferably −60° C. higher, and more preferably −55° C. higher. The temperature is preferably from −60° C. to −20° C., most preferably from −55° C. to −50° C., and specifically −50° C. While benzidine rearrangement reactions was conventionally carried out at temperature from −10 to +80° C., in particular, in the vicinity of −10° C. to 0° C., it has been discovered that the yield is improved by carrying out the benzidine rearrangement reaction under the low temperature conditions according to the present invention. By making the temperature extremely low as stated above, the removal of heat is effectively performed and side reactions are suppressed during the rearrangement reaction so that the selectivity of the rearrangement reaction improves.

More preferably, in the preparation method of the present invention, an additive is added, which prevents the solidification of the reaction mixture and/or improves the flowability of the reaction mixture in the benzidine rearrangement reaction. By carrying out the benzidine rearrangement reaction in the presence of the additive, it is possible to prevent increase in the viscosity of the reaction solution so as to avoid decrease in the flowability, and to prevent the solidification of the reaction solution so as to avoid difficulty in stirring in the low temperature conditions stated above, specifically, in temperature conditions of from −70° C. to −45° C., preferably from −55° C. to −50° C. Namely, by carrying out the benzidine rearrangement reaction in the presence of the additive, the flowability of the reaction solution is not impaired and stirring is performed well even under the aforementioned extremely low temperature conditions. By using the additive at a temperature of from −70° C. to −45° C., even at higher than −45° C. and up to −11° C., the stirring of the reaction solution is made easier to increase the efficiency of contact, so that the benzidine rearrangement reaction is completed in an extremely short time, preferably from 1 minute to 1 hour.

The additive is not specifically limited as long as it prevents the solidification of the reaction mixture and/or improves the flowability and improves the stirring of the reaction mixture under the low temperature reaction conditions of the present invention. Examples of the additive include at least from surfactants, monohydric alcohols, dihydric alcohols (glycol), trihydric alcohols (glycerin), ether solvents, glycol ether solvents, carboxylic acid solvents, nitrogen solvents, sulfur solvents, and fluorine solvents. Preferred are surfactants, monohydric alcohols, dihydric alcohols, trihydric alcohols, ether solvents, and glycol ether solvents. Surfactants, monohydric alcohols, and dihydric alcohols are more preferable, and surfactants are even more preferable.

Examples of the surfactant include cationic surfactants, anionic surfactants, amphoteric surfactants, and nonionic surfactants. More specifically, examples of the cationic surfactant include quaternary ammonium salts and fatty acid amidoamines. Examples of the anionic surfactant include a polyoxyethylene styrenated phenyl ether sulfate, polyoxyethylene alkyl ether sulfate, polyoxyethylene alkyl ether phosphate, polyoxyethylene alkyl ether acetate, polyoxyethylene alkyl sulfosuccinate, sulfonates, sulfosuccinate, alkyl sulfates, and fatty acid salts. Examples of the amphoteric surfactant include alkylbetaine, fatty acid amidebetaine, and alkylamine oxides. Examples of the nonionic surfactant include polyoxyalkylene alkyl ethers, polyoxyethylene derivatives, polyoxyethylene-polyoxypropylene glycol, polyoxyethylene alkyl ethers, polyoxyethylene fatty acid esters, alkylalkanolamide, polyoxyethylene hardened castor oil ether, polyoxyethylene alkylamine, sorbitan fatty acid ester, polyoxyethylene sorbitan fatty acid ester, alkyl polyglucoside, sucrose fatty acid ester, and sucrose derivatives. Among them, a nonionic surfactant is preferable, and a polyoxyethylene derivative is more preferable. Surfactants other than those listed above may be used, as long as the surfactant can prevent the solidification of the reaction solution or can improve flowability.

Examples of the additives other than the above-mentioned surfactants include monohydric alcohols, dihydric alcohols (glycol), trihydric alcohols (glycerin), ether solvents, glycolether solvents, carboxylic acid solvents, nitrogen solvents, sulfur solvents, and fluorine solvents. More specifically, examples of the monohydric alcohol include methanol, ethanol, and isopropanol. Examples of the trihydric alcohol(glycerin) include glycerin. Examples of the dihydric alcohol(glycol) include ethylene glycol, diethylene glycol, and propylene glycol. Examples of the ether solvents include tetrahydrofuran. Examples of the glycolether solvents include methyl cellosolve, ethyl cellosolve, and butyl cellosolve. Examples of carboxylic acid solvents include acetic acid. Examples of the nitrogen solvents include 1,3-dimethyl-2-imidazolidinone. Examples of the sulfur solvents include sulfolane. Examples of the fluorine solvents include trifluoromethanesulfonic acid. Among them, methanol and ethylene glycol are preferable, and methanol is more preferable. Water-soluble solvents other than those listed above may be used, as long as the solvent prevents the solidification or can improve the flowability of the reaction solution.

The amount of the additive such as a surfactant or an alcohol in the benzidine rearrangement reaction of the present invention may be 1 to 30 parts by mass, preferably 3 to 10 parts by mass, per 100 parts by mass of the hydrazo compound. By adding an additive within this range, it is possible to effectively suppress the increase of the viscosity of the reaction mixture even under the low temperature conditions of the present invention.

More specifically, an example of the procedures of the benzidine rearrangement reaction in the present invention includes steps of charging the hydrazobenzene compound represented by the formula (2), an organic solvent and an inorganic acid into a flask equipped with a stirrer and stirring for 1 hour to 5 hours under the aforementioned range of reaction temperature to carry out the rearrangement reaction. The reaction temperature is as stated above. In an embodiment in which an additive is not used, the reaction temperature may be more than −50° C., preferably more than −50° C. to −11° C., even more preferably more than −45° C. to −18° C., and still more preferably from −40° C. to −20° C. The reaction time is often appropriately adjusted so that the benzidine rearrangement reaction is completed.

An example of other embodiments of the present invention includes steps of charging the hydrazobenzene compound represented by the formula (2), an organic solvent, an inorganic acid, and an additive such as a surfactant or an alcohol into a flask equipped with a stirrer and stirring for 1 minute to 1 hour under the aforementioned reaction temperature to carry out the rearrangement reaction. The reaction temperature is as stated above. In an embodiment in which an additive is used, the reaction temperature may be from −70° C. to −20° C., preferably from −70° C. to −45° C., more preferably from −60° C. to −48° C., and even more preferably from −55° C. to −50° C. In the embodiment in which the additive is used, the reaction may be completed in an extremely short time. Namely, the reaction time may be 1 minute to 1 hour, preferably 3 to 10 minutes, and specifically 4 to 6 minutes.

The organic solvent is not specifically limited, and may be those used in a conventional benzidine rearrangement reaction. The organic solvent is preferably one having a melting point so as not to solidify under a temperature from −75° C. to −11° C. according to the present invention. The melting point of the solvent is more preferably −25° C. or less, and the melting point is even more preferably −75° C. or less. The organic solvent is preferably a water-immiscible organic solvent, and is different from the water-soluble solvent as the above-mentioned additive. Among them, toluene, ortho-xylene, meta-xylene, chlorobenzene, and ortho-chlorotoluene are preferable, and toluene is more preferable. The amount of organic solvent may be such that the concentration of the hydrazo compound is 15 to 30% by weight, preferably 18 to 27% by weight, and more preferably 20 to 25% by weight. Sulfuric acid and concentrated hydrochloric acid are preferably used as the inorganic acid. In particular, it is preferable to use an aqueous sulfuric acid having a concentration of 55 to 65% by weight, preferably 60% by weight. The amount of the inorganic acid may be 10 to 20 mol, preferably 13 to 17 mol, and more preferably 15 mol, per mol of the hydrazo compound.

The reaction solution may be treated after the rearrangement reaction according to a conventional method. For example, after the reaction, water is added to the reaction solution, the temperature is raised, and the toluene layer is removed. The remaining layer is filtrated, isolated sulfate is neutralized with an aqueous solution of sodium hydroxide and isolating the product to obtain the target compound represented by formula (1).

The hydrazobenzene compound having substituent groups (X₁ and X₂) at the metal position represented by formula (2), more preferably, 3,3′-bis(trifluoromethyl) hydrazobenzene in which X₁ and X₂ are —CF₃ may be prepared by any manner such as a conventional method.

According to the preparation method of the present invention, the diaminobiphenyl compound having a bulky substituent group at the meta position, preferably 2,2′-bis(trifluoromethyl)-4,4′-diaminobiphenyl, is obtained in a yield of 35% or more, preferably 38% to 55%, and in particular, at a high yield of 40% to 50%. The yield is higher compared to the yield of 10% to 30% which is obtained by a conventional preparation method. The closer the temperature to a range of from −11° C. to −70° C., preferably from −20° C. to −50° C., the higher the yield. In the present invention, the yield of the diaminobiphenyl compound is based on the hydrazo compound. In particular, according to the preparation method of the present invention, the reaction time is significantly reduced, compared to a conventional preparation method.

The preferred embodiment of the present invention provides a preparation method of 2,2′-bis(trifluoromethyl)-4,4′-diaminobiphenyl. The compound is useful as a raw material for polyimide resin and polyamide resins.

### EXAMPLES

The present invention will be explained in more detail below by way of the Examples. However, the present invention is not limited to the following Examples.

### Example 1

134 Grams of toluene, 140 g of a 55% sulfuric acid, and 23.6 g of 3,3′-bis(trifluoromethyl) hydrazobenzene were placed in a 300 ml flask equipped with an Allihn condenser, a thermometer, and a stirrer and allowed to react at −20° C. for 5 hours. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C. and filtered to obtain a cake. The cake was dried overnight at 70° C. and 9.0 g of a product was obtained. ¹H-NMR analysis determined that the product was 2,2′-bis(trifluoromethyl)-4,4′-diaminobiphenyl represented by the formula (I). The yield was 38%, the conversion was 97%, and the selectivity was 59%.

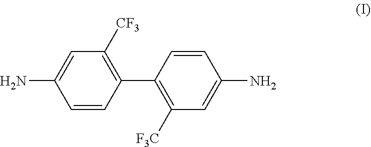 [SMILES: Nc1ccc(-c2ccc(N)cc2C(F)(F)F)c(C(F)(F)F)c1]

### Example 2

134 Grams of toluene, 140 g of a 55% sulfuric acid, and 23.6 g of 3,3′-bis(trifluoromethyl) hydrazobenzene were placed in a 300 ml flask equipped with an Allihn condenser, a thermometer, and a stirrer, and allowed to react at −40° C. for 5 hours. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 7.6 g of a product was obtained. ¹H-NMR analysis determined that the product was 2,2′-bis(trifluoromethyl)-4,4′-diaminobiphenyl represented by the aforesaid formula (I). The yield was 46%, the conversion was 97%, and the selectivity was 71%.

### Example 3

134 Grams of toluene, 140 g of a 55% sulfuric acid, 23.6 g of 3,3′-bis(trifluoromethyl) hydrazobenzene, and 1.2 g of a nonionic surfactant which was a polyoxyethylene derivative were placed in a 300 ml flask equipped with an Allihn condenser, a thermometer, and a stirrer, and allowed to react at −20° C. for 5 minutes. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 9.0 g of a product was obtained. ¹H-NMR analysis determined that the product was 2,2′-bis(trifluoromethyl)-4,4′-diaminobiphenyl represented by the aforesaid formula (I). The yield was 40%, the conversion was 97%, and the selectivity was 62%.

### Example 4

80 Grams of toluene, 176 g of a 60% sulfuric acid, 23.6 g of 3,3′-bis(trifluoromethyl) hydrazobenzene, and 1.2 g of a nonionic surfactant which was a polyoxyethylene derivative were placed in a 300 ml flask equipped with an Allihn condenser, a thermometer, and a stirrer, and allowed to react at −50° C. for 5 minutes. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 11.8 g of a product was obtained. ¹H-NMR analysis determined that the product was 2,2′-bis(trifluoromethyl)-4,4′-diaminobiphenyl represented by the aforesaid formula (I). The yield was 50%, the conversion was 97%, and the selectivity was 77%.

### Example 5

80 Grams of toluene, 176 g of a 60% sulfuric acid, 23.6 g of 3,3′-bis(trifluoromethyl) hydrazobenzene, and 1.2 g of a cationic surfactant which was a quaternary ammonium salts were placed in a 300 ml flask equipped with an Allihn condenser, a thermometer, and a stirrer, and allowed to react at −50° C. for 5 minutes. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 10.8 g of a product was obtained. ¹H-NMR analysis determined that the product was 2,2′-bis(trifluoromethyl)-4,4′-diaminobiphenyl represented by the aforesaid formula (I). The yield was 46%, the conversion was 90%, and the selectivity was 76%.

### Example 6

80 Grams of toluene, 176 g of a 60% sulfuric acid, 23.6 g of 3,3′-bis(trifluoromethyl) hydrazobenzene, and 1.2 g of an anionic surfactant which was a sulfonate were placed in a 300 ml flask equipped with an Allihn condenser, a thermometer, and a stirrer, and allowed to react at −50° C. for 5 minutes. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 10.5 g of a product was obtained. ¹H-NMR analysis determined that the product was 2,2′-bis(trifluoromethyl)-4,4′-diaminobiphenyl represented by the aforesaid formula (I). The yield was 45%, the conversion was 90%, and the selectivity was 74%.

### Example 7

80 Grams of toluene, 176 g of a 60% sulfuric acid, 23.6 g of 3,3′-bis(trifluoromethyl) hydrazobenzene, 1.2 g of an amphoteric surfactant which was an alkyl betaine were placed in a 300 ml flask equipped with an Allihn condenser, a thermometer, and a stirrer, and allowed to react at −50° C. for 5 minutes. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 10.8 g of a product was obtained. ¹H-NMR analysis determined that the product was 2,2′-bis(trifluoromethyl)-4,4′-diaminobiphenyl represented by the aforesaid formula (I). The yield was 46%, the conversion was 92%, and the selectivity was 74%.

### Example 8

80 Grams of toluene, 176 g of a 60% sulfuric acid, 23.6 g of 3,3′-bis(trifluoromethyl) hydrazobenzene, and 1.2 g of methanol were placed in a 300 ml flask equipped with an Allihn condenser, a thermometer, and a stirrer, and allowed to react at −50° C. for 5 minutes. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 10.6 g of a product was obtained. ¹H-NMR analysis determined that the product was 2,2′-bis(trifluoromethyl)-4,4′-diaminobiphenyl represented by the aforesaid formula (I). The yield was 45%, the conversion was 92%, and the selectivity was 73%.

### Example 9

80 Grams of toluene, 176 g of a 60% sulfuric acid, 23.6 g of 3,3′-bis(trifluoromethyl) hydrazobenzene, and 1.2 g of ethylene glycol were placed in a 300 ml flask equipped with an Allihn condenser, a thermometer, and a stirrer, and allowed to react at −50° C. for 5 minutes. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 10.5 g of a product was obtained. ¹H-NMR analysis determined that the product was 2,2′-bis(trifluoromethyl)-4,4′-diaminobiphenyl represented by the aforesaid formula (I). The yield was 44%, the conversion was 92%, and the selectivity was 72%.

### Comparative Example 1

134 Grams of toluene, 140 g of a 55% sulfuric acid, and 23.6 g of 3,3′-bis(trifluoromethyl) hydrazobenzene were placed in a 300 ml flask equipped with an Allihn condenser, a thermometer, and a stirrer, and allowed to react at 0° C. for 5 hours. After the reaction, water was added and heated to 70° C. The toluene layer was then removed. The sulfuric acid layer was cooled to cause crystallization and filtered to obtain a sulfate cake. The sulfate cake and water were placed in a 300 ml flask equipped with an Allihn condenser, a thermometer, and a stirrer, and neutralized by adding aqueous NaOH while maintaining the temperature at 80° C. The slurry was cooled to 50° C., filtered to obtain a cake. The cake was dried overnight at 70° C. and 7.6 g of a product was obtained. ¹H-NMR analysis determined that the product was 2,2′-bis(trifluoromethyl)-4,4′-diaminobiphenyl represented by the aforesaid formula (I). The yield was 32%, the conversion was 97%, and the selectivity was 49%.

The additives, the reaction temperatures, the reaction times, the sulfuric acid concentrations, and the yields in the method of Examples 1 to 9 and Comparative example 1 are summarized in the following Table 1.

TABLE 1

| | | Reaction | | Sulfuric acid | |
|---|---|---|---|---|---|
| | | temperature | Reaction | concentration | Yield |
| | Additive | ° C. | time | % | % |
| Example 1 | None | −20 | 5 hours | 55 | 38 |
| Example 2 | None | −40 | 5 hours | 55 | 46 |
| Example 3 | Nonionic | −20 | 5 minutes | 55 | 40 |
| | surfactant | | | | |
| Example 4 | Nonionic | −50 | 5 minutes | 60 | 50 |
| | surfactant | | | | |
| Example 5 | Cationic | −50 | 5 minutes | 60 | 46 |
| | surfactant | | | | |
| Example 6 | Anionic | −50 | 5 minutes | 60 | 45 |
| | surfactant | | | | |
| Example 7 | Amphoteric | −50 | 5 minutes | 60 | 46 |
| | surfactant | | | | |
| Example 8 | Methanol | −50 | 5 minutes | 60 | 45 |
| Example 9 | Ethylene | −50 | 5 minutes | 60 | 44 |
| | glycol | | | | |
| Comparative | None | 0 | 5 hours | 55 | 32 |
| example 1 | | | | | |

As shown in Table 1, the yield was 32% and the selectivity was 49% by the preparation method of Comparative example 1 in which the reaction temperature was 0° C. In contrast, the yield was 38% and the selectivity was 59% by the preparation method of Example 1 in which the reaction temperature was −20° C.; the yield was 46% and the selectivity was 71% by the preparation method of Example 2 in which the reaction temperature was −40° C., and the yield was 50% and the selectivity was 77% by the preparation method of Example 4 in which the reaction temperature was −50° C. As seen in the results, the closer the reaction temperature is made to a low temperature from −20° C. to −50° C., the more the selectivity may increase, and the greater the improvement of the yield.

As seen in Examples 3 to 9, the reaction is completed in an extremely short period of time with a reaction time of about 5 minutes due to the addition of a surfactant, methanol or ethylene glycol. This is because the addition of the specific additives of the present application such as a surfactant improves the flowability of the reaction solution and, thereby, stirring becomes easier and the efficiency of contact in the rearrangement reaction is improved.

According to the preparation method of the present invention, the diaminobiphenyl compound, specifically, a 2,2′-bis(trifluoromethyl)-4,4′-diaminobiphenyl is preferably produced in a high yield, and the shortening of the reaction time is possible and, therefore, the productivity is greatly improved which is useful.

1. A method for preparing a diaminobiphenyl compound represented by the following formula (1): 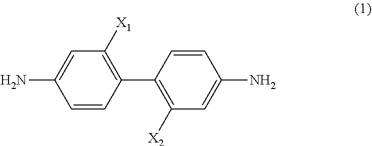
wherein X₁ and X₂ are, independently of each other, a group selected from the group consisting of a trifluoromethyl group and, optionally fluorinated, isopropyl, isobutyl, sec-butyl, tert-butyl and neopentyl groups,
comprising a step of subjecting a diphenylhydrazine compound represented by the following formula (2) 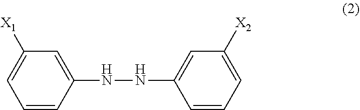
wherein X₁ and X₂ are as defined above,
to a benzidine rearrangement reaction in the presence of an organic solvent and an inorganic acid at a temperature of from −70° C. to −11° C.
to obtain the diaminobiphenyl compound represented by the formula (1).

2. The method according to claim 1, wherein the benzidine rearrangement reaction is carried out at a temperature of from −60° C. to −20° C.

3. The method according to claim 1, wherein X₁ and X₂ in the formulas (1) and (2) are trifluoromethyl groups.

4. The method according to claim 1, wherein the benzidine rearrangement reaction is carried out in the presence of an additive for preventing solidification of a reaction mixture and/or improving flowability of the reaction mixture.

5. The method according to claim 4, wherein the benzidine rearrangement reaction is carried out in the presence of the additive for a reaction time of 1 minute to 1 hour, at the temperature of from −70° C. to −11° C.

6. The method according to claim 4, wherein the benzidine rearrangement reaction is carried out in the presence of the additive at a temperature of from −70° C. to −45° C.

7. The method according to claim 6, wherein the benzidine rearrangement reaction is carried out at a temperature of from −55° C. to −50° C.

8. The method according to claim 4, wherein the additive is at least one selected from surfactants, monohydric alcohols, dihydric alcohols, trihydric alcohols, ether solvents, glycol ether solvents, carboxylic acid solvents, nitrogen solvents, sulfur solvents, and fluorinated solvents.

9. The method according to claim 8, wherein the additive is at least one selected from cationic surfactants, anionic surfactants, amphoteric surfactants, and nonionic surfactants.

10. The method according to claim 9, wherein the additive is a nonionic surfactant.

11. The method according to claim 2, wherein X₁ and X₂ in the formulas (1) and (2) are trifluoromethyl groups.

12. The method according to claim 2, wherein the benzidine rearrangement reaction is carried out in the presence of an additive for preventing solidification of a reaction mixture and/or improving flowability of the reaction mixture.

13. The method according to claim 3, wherein the benzidine rearrangement reaction is carried out in the presence of an additive for preventing solidification of a reaction mixture and/or improving flowability of the reaction mixture.

14. The method according to claim 4, wherein the benzidine rearrangement reaction is carried out in the presence of the additive for a reaction time of 1 minute to 1 hour, at a temperature of from −60° C. to −20° C.

15. The method according to claim 5, wherein the benzidine rearrangement reaction is carried out in the presence of the additive at a temperature of from −70° C. to −45° C.

16. The method according to claim 15, wherein the benzidine rearrangement reaction is carried out at a temperature of from −55° C. to −50° C.

17. The method according to claim 16, wherein the additive is at least one selected from surfactants, monohydric alcohols, dihydric alcohols, trihydric alcohols, ether solvents, glycol ether solvents, carboxylic acid solvents, nitrogen solvents, sulfur solvents, and fluorinated solvents.

18. The method according to claim 5, wherein the additive is at least one selected from surfactants, monohydric alcohols, dihydric alcohols, trihydric alcohols, ether solvents, glycol ether solvents, carboxylic acid solvents, nitrogen solvents, sulfur solvents, and fluorinated solvents.

19. The method according to claim 6, wherein the additive is at least one selected from surfactants, monohydric alcohols, dihydric alcohols, trihydric alcohols, ether solvents, glycol ether solvents, carboxylic acid solvents, nitrogen solvents, sulfur solvents, and fluorinated solvents.

20. The method according to claim 7, wherein the additive is at least one selected from surfactants, monohydric alcohols, dihydric alcohols, trihydric alcohols, ether solvents, glycol ether solvents, carboxylic acid solvents, nitrogen solvents, sulfur solvents, and fluorinated solvents.
